(19) United States
(12) Patent Application Publication
(10) Pub. No.: US 2021/0145372 A1
(43) Pub. Date: May 20, 2021

(54) IMAGE ACQUISITION BASED ON TREATMENT DEVICE POSITION

(71) Applicant: Brainlab AG

(72) Inventors: Kajetan Berlinger, Munich (DE); Stefan Seifert, Isen (DE)

(21) Appl. No.: 16/466,383

(22) PCT Filed: Jun. 12, 2018
(86) PCT No.: PCT/EP2018/065518

§ 371 Date: Jun. 4, 2019

(51) Int. Cl. A61B 6/02; G06T 7/00; A61B 6/04; A61B 6/00

(52) U.S. Cl. CPC A61B 6/022; A61B 6/547; A61B 6/0492; G06T 7/0012

(57) ABSTRACT

The disclosed method encompasses acquiring position data. The position data describes predetermined positions of a treatment device. At each of the predetermined positions, an imaging condition is fulfilled. Such an imaging condition is for a free line of sight of two (stereo-)imaging units at the same time. In a next step, the current position of the treatment device is acquired. Then, the current position is compared with the predetermined positions. In case the current position corresponds to a predetermined position, decision data is determined which describes that an image shall be taken. In a next step, control data is determined which describes a control signal for an imaging device to take an image or not to take an image, depending on the decision data.

### FIELD OF THE INVENTION

The present invention relates to a computer-implemented method for determining control data describing a control signal for controlling an imaging device to take an image or not to take an image, a corresponding computer program, a non-transitory program storage medium storing such a program and a computer for executing the program, as well as a medical system comprising an electronic data storage device and the aforementioned computer.

### TECHNICAL BACKGROUND

Some medical systems using image guidance enable the acquisition of X-ray images at any point in time, for example during a treatment session. In the field of image guided surgery or therapy, until now, images had to be acquired based on user input. For example, a user had to decide at which position of a treatment device and at which point in time an image should be taken. The user then pressed a button to acquire the image. Other medical systems using image guidance can't be used to monitor non-coplanar treatments as the imager (imaging unit) would in this case collide with the patient support device (couch).

This known approach (manual image acquisition) has the disadvantage that several circumstances might lead to an image with a bad image quality, for example in case the line of sight of the imaging device is obstructed during the acquisition of the image, e.g. by parts of a gantry of the treatment device. Also, other factors can lead to a loss of image quality, e.g. a collision of the imaging device with other devices. Furthermore, the point in time at which the user acquires an image is not always the best. That is, for the purpose of determining a position of an object (e.g. a patient), certain imaging angles or points in time are better than others. Also, unnecessary images might be taken, e.g. during an obstructed view (image acquired by accident using obstructed imager), in case no stereo view is available, in case no dose is currently applied, in case the reconstructability (3D) using the acquired images is not possible or leads to bad results, or in case the acquired images are acquired at a closely similar line of sight (in this case the images can't be combined to reliably reconstruct a position in space).

Automatically triggering the image acquisition is important, as the monitoring system should know at what point in time an image of high value (to enable a physician to obtain a clinical decision) can be acquired. In some cases, the point in time of acquisition is also very important as the gantry might obstruct the line of sight of one imager. Previous system did not acquire any X-ray images automatically. The current application therefore considers automatically triggered image acquisition, i.e. aims at (automatically) determining control data describing a control signal for controlling an imaging device to take an image or not to take an image, considering the above.

The present invention can be used with treatment devices such as radiation treatment devices used for radiation therapy or radiation surgery. For example, it can be used e.g. in connection with a system for image-guided radiotherapy such as VERO® and ExacTrac®, both products of Brainlab AG.

Aspects of the present invention, examples and exemplary steps and their embodiments are disclosed in the following. Different exemplary features of the invention can be combined in accordance with the invention wherever technically expedient and feasible.

### EXEMPLARY SHORT DESCRIPTION OF THE INVENTION

In the following, a short description of the specific features of the present invention is given which shall not be understood to limit the invention only to the features or a combination of the features described in this section.

The disclosed method encompasses acquiring position data. The position data describes predetermined positions of a treatment device such as a radiation treatment apparatus. These predetermined positions are not randomly selected. Instead, at each of the predetermined positions, an imaging condition is fulfilled. Such an imaging condition is for example a free line of sight of an imaging unit, a free line of sight of two (stereo-)imaging units at the same time, a certain position on an arc of a patient treatment plan (e.g. the middle or the start/end point), or else. These imaging conditions guarantee that an image with a high quality can be acquired, for example without any artefacts caused by the gantry lying in the line of sight of the imaging device.

In a next step, the current position of the treatment device is acquired (described by status data).

Then, the current position is compared with the predetermined positions. In case the current position corresponds (e.g. is equal to or lies within a certain range) to a predetermined position, decision data is determined which describes that an image shall be taken. At this current position, the imaging condition is fulfilled. In case the current position does not correspond to a predetermined position, the decision data describes the opposite, i.e. that no image shall be taken.

In a next step, control data is determined which describes a control signal for an imaging device to take an image or not to take an image, depending on the decision data.

As a consequence, during movement of the treatment device, images can be acquired automatically at advantageous positions of the treatment device without a user having to manually trigger the image acquisition.

### GENERAL DESCRIPTION OF THE INVENTION

In this section, a description of the general features of the present invention is given for example by referring to possible embodiments of the invention.

In general, the invention reaches the aforementioned object by providing, in a first aspect, a computer-implemented medical method medical data processing method for determining control data. The control data describes a control signal for controlling an imaging device to take an image or not to take an image. The method comprises executing, on at least one processor of at least one computer (for example at least one computer being part of a navigation system or of a (radiation) treatment system), the following exemplary steps which are executed by the at least one processor.

In a (for example first) exemplary step, position data is acquired. For example, the position data describes at least one predetermined position of a radiation treatment device. For example, the predetermined position is defined in a first reference system. For example, the predetermined position is defined relative to a normal position of the treatment device, i.e. an isocenter of an imaging device which is positioned in a predetermined positional relationship to the treatment device. The predetermined position is for example defined relative to a position of a patient, for example relative to a position of an anatomical body part of a patient. The patient is for example positioned on a patient positioning device such as a patient couch. The patient positioning device is for example positioned in a predetermined positional relationship to the treatment device. The patient is for example positioned in a predetermined positional relationship to the treatment device. Alternatively, or additionally, the position of the patient in relation to the treatment device is determined, for example using known image-based registration methods (e.g. image-fusion based mapping using markers and/or a marker device and/or a reference star and for example a surgical navigation system). The position of the patient may alternatively or additionally be determined using patient tracking methods (e.g. optical tracking of a marker attached to a predetermined anatomical body part of the patient).

For example, at least one imaging condition must be fulfilled in case the radiation treatment device is positioned in any of the at least one predetermined position. Note that when acquiring the position data, the treatment device may be positioned in any position. In case the treatment device at some point in time reaches one of the at least one predetermined position, the at least one imaging condition is fulfilled.

In a (for example second) exemplary step, status data is acquired. For example, the status data describes a current position of the radiation treatment device. The current position is for example described in a second reference system which lies in a predetermined positional relationship to the treatment device or to the patient. For example, the second reference system is equal to the first reference system. In any case, the first reference system and the second reference system need to be in a predetermined positional relationship. For example, a transformation matrix describing a transformation between the first and the second reference system is known (e.g. predetermined). In case it is in this application referred to a predetermined positional relationship, a transformation matrix describing a transformation from a first position to a second position in a given reference system may be used to describe the predetermined positional relationship. In case of different reference systems, an additional transformation matrix describing a transformation between the first and the second reference system can be used to describe a predetermined positional relationship between a first point in a first reference system and a second point in a second reference system. The current position is for example given as one or more of a point, an angle, a plane or a line in the second reference system.

In a (for example third) exemplary step, decision data is determined based on the position data and the status data. For example, in case it is determined that the current position described by the status data corresponds to (i.e. is equal to and/or lies within a predetermined (e.g. based on user input) positional range in relation to) one of the at least one predetermined position described by the position data, the decision data is determined such that it describes that an image shall be taken by the imaging device. For example, in case it is determined that the current position described by the status data does not correspond to (i.e. is not equal to and/or lies outside a predetermined (e.g. based on user input) positional range in relation to) one of the at least one predetermined position described by the position data, the decision data is determined such that it describes that no image shall be taken by the imaging device.

In a (for example fourth) exemplary step, control data is determined based on the decision data. For example, in case the decision data describes that an image shall be taken by the imaging device, the control data is determined such that it describes a control signal for controlling the imaging device to take an image. In this case, the control signal may be represented as “1”, “TRUE”, “ON”, a positive command signal (such as a trigger signal) or else. For example, in case the decision data describes that no image shall be taken by the imaging device, the control data is determined such that it describes a control signal for controlling the imaging device not to take an image. In this case, the control signal may be represented as “0”, “FALSE”, “OFF”, an empty signal, a negative command signal (such as no signal at all or a negative trigger signal), or else.

In an example, the imaging device (e.g. an analytical imaging device as described below) comprises an imaging unit (e.g. an x-ray imaging unit, a cone-beam CT imaging unit or else). In this example, one of the at least one imaging condition is fulfilled in case the imaging unit has a free line of sight.

For example, the line of sight runs between an emitter and a receiver comprised in the imaging device and/or comprised in the imaging unit. The emitter and the receiver are for example configured to capture an image, for example an image of a patient. The line of sight is for example a volume between the emitter and the receiver. The line of sight is for example a volume between the emitter and the receiver which is subject to image acquisition, i.e. which is imaged with the imaging device (i.e. the imaging unit). In this example, the line of sight is the volume within the field of view of the imaging device (i.e. the imaging unit). For example, matter which lies within the line of sight of the imaging device is imaged by the imaging device when acquiring an image, whilst matter which does not lie within the line of sight of the imaging device is not imaged by the imaging device when acquiring the image. That is, in an example, only matter which lies within the line of sight of the imaging device is imaged by the imaging device when acquiring an image.

For example, the imaging unit has a free line of sight in case no unwanted devices obstruct the line of sight of the imaging unit (a line of sight between an emitter and a receiver comprised in the imaging unit, wherein the emitter and the receiver are configured to capture an image). These unwanted devices can be positioned outside the line of sight by moving the unwanted devices and/or (at least a part of) the imaging unit without limiting the function of a treatment device (e.g. limiting or prohibiting the radiation of an anatomical body part of a patient). For example, the unwanted device is a part of a gantry of a treatment device. For example, the imaging unit has a free line of sight in case a predetermined portion (i.e. a predetermined region or a predetermined minimum areal amount) of the acquired image is free of the unwanted devices. For example, the imaging unit has a free line of sight in case a predetermined portion (i.e. a predetermined spatial region or a predetermined minimum volumetric amount) of the line of sight is free of the unwanted devices.

The imaging unit has a free line of sight even if some material obstructs the line of sight of the imaging device, in case the some material cannot be positioned outside the line of sight without limiting the function of a treatment device (e.g. limiting or prohibiting the radiation of an anatomical body part of a patient) and/or worsening the required/intended imaging results (e.g. limiting or prohibiting the imaging of a relevant anatomical body part of a patient). For example, the some material is a patient positioning device (e.g. a patient couch) and/or the patient and/or a marker and/or a marker device and/or a reference star.

In an example, the imaging device comprises two imaging units, for example arranged such that a stereoscopic image can be obtained with the imaging device. In this example, one of the at least one imaging condition is fulfilled in case each of the two imaging units has a free line of sight (at the same point in time). For example, each of the two imaging units has a position which does not change in case the treatment device changes its position, for example each of the two imaging units has a fixed spatial position.

In an example, at least two imaging conditions must be fulfilled in case the radiation treatment device is positioned in any of the at least one predetermined position. In this example, one of the at least two imaging conditions is fulfilled in case the predetermined position corresponds to a first position which fulfills a predetermined positional relationship to a second position.

The first position fulfills a predetermined positional relationship to a second position for example in case both positions are spaced (approximately) 90° (e.g. more than 70°, more than 80°, more than 85°, more than 89° and/or less than 91°, less than 95°, less than 100°, less than 110°, these ranges being for example set by a user) apart from one another. That is, a theoretical treatment beam to be emitted by the treatment device at the first position is (approximately) perpendicular to a theoretical treatment beam to be emitted by the treatment device at the second position (e.g. angle of intersection of both theoretical treatment beams is more than 70°, more than 80°, more than 85°, more than 89° and/or less than 91°, less than 95°, less than 100°, less than 110°, these ranges or limits being for example set by a user).

Another of the at least two imaging conditions is for example fulfilled in case one imaging unit comprised in the imaging device has a free line of sight. Another of the at least two imaging conditions is for example fulfilled in case two imaging units comprised in the imaging device both have a free line of sight (at the same point in time).

For example, the second position corresponds to another one of the at least one predetermined position. That is, in case the treatment device is positioned in the first position both of the at least two imaging conditions are fulfilled, and in case the treatment device is positioned in the second position both of the at least two imaging conditions are fulfilled.

For example, one of the at least one imaging condition is fulfilled in case the predetermined position corresponds to a certain position on a treatment arc defined in a patient treatment plan. For example, the certain position is a predetermined position (e.g. user-defined) described in relation to any arc (e.g. a middle of an arc, an end of an arc or else). For example, the patient treatment plan is described by patient treatment plan data. For example, the first position and the second position both lie on an arc. The arc is for example defined by a plurality of positions of the treatment device, for example a plurality of positions which lie in a given plane. For example, the arc is a continuous line on which lies a plurality of positions of the treatment device. The arc for example describes a segment of an ellipse, for example a segment of a circle. The arc is for example defined in the first reference system (or in the second reference system). For example, patient treatment plan data is acquired which describes at least one arc. For example, the arc defines a plurality of positions of the treatment device (and/or of a theoretical treatment beam to be emitted by the treatment device, i.e. a treatment beam arrangement) in a temporal order. For example, the arc defines how the treatment device shall move during a treatment. Any known method to determine the patient treatment plan data may be used. For example, the arc is determined based on user input.

For example, the treatment arc is divided into a plurality of arc-segments. For example, the certain position is a position defining a boundary point of an arc-segment (e.g. a position between two arc-segments, a position at the start or at the end of an arc-segment and/or a position at the start or end of the arc).

For example, the plurality of arc-segments comprises or consists of two or more arc-segments of equal size, for example (of) a number N of arc-segments. This number N may be predetermined, user-defined or determined depending on the size of the arc and a predetermined (e.g. user-defined) arc-segment size such as, for example, 5°, 10°, 12°, 15°, 50° or else. Note that (not only, but also) in case of arcs which define a plurality of positions of the treatment device which do not lie in the same plane, alternative arc-sizes and sizes of arc-segments may be used, e.g. described by several numbers (e.g. in polar coordinates) instead of simple values of degrees.

For example, one of the at least one imaging condition is fulfilled in case the predetermined position corresponds to a position at which no treatment beam is to be emitted by the treatment device. For example, the patient treatment plan data describes positions at which a treatment beam is to be emitted by the treatment device. For example, the patient treatment plan data describes positions at which a treatment beam is not to be emitted by the treatment device.

For example, one of the at least one imaging condition is fulfilled in case the predetermined position corresponds to a position at which the imaging device can take an image. For example, this is a position at which the imaging device does not collide with any other device (such as a gantry of the treatment device, a patient support device or else). For example, this is a position at which all components of the imaging device are functional, for example arranged in an imaging configuration (e.g. detector panel and/or emitter of the imaging unit arranged in an image acquisition position).

For example, one of the at least one imaging condition is fulfilled in case the predetermined position corresponds to a position with a predetermined positional relationship (e.g. a given distance and/or a given angle difference) to a collision position. For example, at the collision position, the imaging device collides with another device (such as a gantry of the treatment device, a patient support device or else).

For example, at least two imaging conditions must be fulfilled in case the radiation treatment device is positioned in any of the at least one predetermined position. In this case, each of the at least two imaging conditions is for example one of the at least one imaging condition described above. Two (or more) imaging conditions can be combined to further enhance the image quality and prevent a damage of the involved devices.

In a second aspect, the invention is directed to a computer program which, when running on at least one processor (for example, a processor) of at least one computer (for example, a computer) or when loaded into at least one memory (for example, a memory) of at least one computer (for example, a computer), causes the at least one computer to perform the above-described method according to the first aspect. The invention may alternatively or additionally relate to a (physical, for example electrical, for example technically generated) signal wave, for example a digital signal wave, carrying information which represents the program, for example the aforementioned program, which for example comprises code means which are adapted to perform any or all of the steps of the method according to the first aspect. A computer program stored on a disc is a data file, and when the file is read out and transmitted it becomes a data stream for example in the form of a (physical, for example electrical, for example technically generated) signal. The signal can be implemented as the signal wave which is described herein. For example, the signal, for example the signal wave is constituted to be transmitted via a computer network, for example LAN, WLAN, WAN, mobile network, for example the internet. For example, the signal, for example the signal wave, is constituted to be transmitted by optic or acoustic data transmission. The invention according to the second aspect therefore may alternatively or additionally relate to a data stream representative of the aforementioned program.

In a third aspect, the invention is directed to a non-transitory computer-readable program storage medium on which the program according to the second aspect is stored.

In a fourth aspect, the invention is directed to at least one computer (for example, a computer), comprising at least one processor (for example, a processor) and at least one memory (for example, a memory), wherein the program according to the second aspect is running on the processor or is loaded into the memory, or wherein the at least one computer comprises the computer-readable program storage medium according to the third aspect.

In a fifth aspect, the invention is directed to a medical system, comprising:
a) the at least one computer according to the fourth aspect;
b) at least one electronic data storage device storing at least the position data, the at least one computer being operably coupled to the at least one electronic data storage device for acquiring, from the at least one data storage device, at least the position data.

In an example of the system according to the fifth aspect, the medical device comprises the treatment device, for example comprising a treatment beam source and a patient support unit (such as at least one of a patient bed or a headrest). The at least one computer is then operably coupled to the treatment device for acquiring, from the treatment device, the status data.

In an example of the system according to the fifth aspect, the medical device comprises the imaging device, for example comprising one or more imaging units. The at least one computer is then operably coupled to the imaging device for controlling the imaging device to take an image or not to take an image based on the control signal described by the control data.

For example, the invention does not involve or in particular comprise or encompass an invasive step which would represent a substantial physical interference with the body requiring professional medical expertise to be carried out and entailing a substantial health risk even when carried out with the required professional care and expertise.

For example, the invention does not comprise a step of performing a treatment of a patient. The invention does not include irradiating a patient for therapeutic or surgical treatment. More particularly, the invention does not involve or in particular comprise or encompass any surgical or therapeutic activity. The invention is instead directed as applicable to determine control data. This data for example describes a control signal for controlling an imaging device to take an image or not to take an image. For this reason alone, no surgical or therapeutic activity and in particular no surgical or therapeutic step is necessitated or implied by carrying out the invention.

The present invention also relates to the use of the device/system or any embodiment thereof for controlling an imaging device to take an image or not to take an image, for example an image of a patient. The use comprises for example at least one of the following steps:
acquiring treatment plan data describing treatment positions of the treatment device, for example from a storage medium or determining the treatment plan data based on user input
defining one or more imaging conditions, e.g. by acquiring the one or more imaging conditions from a storage medium or determining the one or more imaging conditions based on user input
conducting the method according to the first aspect to determine the control data.

### DEFINITIONS

In this section, definitions for specific terminology used in this disclosure are offered which also form part of the present disclosure.

### Computer Implemented Method

The method in accordance with the invention is for example a computer implemented method. For example, all the steps or merely some of the steps (i.e. less than the total number of steps) of the method in accordance with the invention can be executed by a computer (for example, at least one computer). An embodiment of the computer implemented method is a use of the computer for performing a data processing method. An embodiment of the computer implemented method is a method concerning the operation of the computer such that the computer is operated to perform one, more or all steps of the method.

The computer for example comprises at least one processor and for example at least one memory in order to (technically) process the data, for example electronically and/or optically. The processor being for example made of a substance or composition which is a semiconductor, for example at least partly n- and/or p-doped semiconductor, for example at least one of II-, III-, IV-, V-, VI-semiconductor material, for example (doped) silicon and/or gallium arsenide. The calculating or determining steps described are for example performed by a computer. Determining steps or calculating steps are for example steps of determining data within the framework of the technical method, for example within the framework of a program. A computer is for example any kind of data processing device, for example electronic data processing device. A computer can be a device which is generally thought of as such, for example desktop PCs, notebooks, netbooks, etc., but can also be any programmable apparatus, such as for example a mobile phone or an embedded processor. A computer can for example comprise a system (network) of “sub-computers”, wherein each sub-computer represents a computer in its own right. The term “computer” includes a cloud computer, for example a cloud server. The term “cloud computer” includes a cloud computer system which for example comprises a system of at least one cloud computer and for example a plurality of operatively interconnected cloud computers such as a server farm. Such a cloud computer is preferably connected to a wide area network such as the world wide web (WWW) and located in a so-called cloud of computers which are all connected to the world wide web. Such an infrastructure is used for “cloud computing”, which describes computation, software, data access and storage services which do not require the end user to know the physical location and/or configuration of the computer delivering a specific service. For example, the term “cloud” is used in this respect as a metaphor for the Internet (world wide web). For example, the cloud provides computing infrastructure as a service (IaaS). The cloud computer can function as a virtual host for an operating system and/or data processing application which is used to execute the method of the invention. The cloud computer is for example an elastic compute cloud (EC2) as provided by Amazon Web Services™. A computer for example comprises interfaces in order to receive or output data and/or perform an analogue-to-digital conversion. The data are for example data which represent physical properties and/or which are generated from technical signals. The technical signals are for example generated by means of (technical) detection devices (such as for example devices for detecting marker devices) and/or (technical) analytical devices (such as for example devices for performing (medical) imaging methods), wherein the technical signals are for example electrical or optical signals. The technical signals for example represent the data received or outputted by the computer. The computer is preferably operatively coupled to a display device which allows information outputted by the computer to be displayed, for example to a user. One example of a display device is a virtual reality device or an augmented reality device (also referred to as virtual reality glasses or augmented reality glasses) which can be used as “goggles” for navigating. A specific example of such augmented reality glasses is Google Glass (a trademark of Google, Inc.). An augmented reality device or a virtual reality device can be used both to input information into the computer by user interaction and to display information outputted by the computer. Another example of a display device would be a standard computer monitor comprising for example a liquid crystal display operatively coupled to the computer for receiving display control data from the computer for generating signals used to display image information content on the display device. A specific embodiment of such a computer monitor is a digital lightbox. An example of such a digital lightbox is Buzz®, a product of Brainlab AG. The monitor may also be the monitor of a portable, for example handheld, device such as a smart phone or personal digital assistant or digital media player.

The invention also relates to a program which, when running on a computer, causes the computer to perform one or more or all of the method steps described herein and/or to a program storage medium on which the program is stored (in particular in a non-transitory form) and/or to a computer comprising said program storage medium and/or to a (physical, for example electrical, for example technically generated) signal wave, for example a digital signal wave, carrying information which represents the program, for example the aforementioned program, which for example comprises code means which are adapted to perform any or all of the method steps described herein.

Within the framework of the invention, computer program elements can be embodied by hardware and/or software (this includes firmware, resident software, micro-code, etc.). Within the framework of the invention, computer program elements can take the form of a computer program product which can be embodied by a computer-usable, for example computer-readable data storage medium comprising computer-usable, for example computer-readable program instructions, “code” or a “computer program” embodied in said data storage medium for use on or in connection with the instruction-executing system. Such a system can be a computer; a computer can be a data processing device comprising means for executing the computer program elements and/or the program in accordance with the invention, for example a data processing device comprising a digital processor (central processing unit or CPU) which executes the computer program elements, and optionally a volatile memory (for example a random access memory or RAM) for storing data used for and/or produced by executing the computer program elements. Within the framework of the present invention, a computer-usable, for example computer-readable data storage medium can be any data storage medium which can include, store, communicate, propagate or transport the program for use on or in connection with the instruction-executing system, apparatus or device. The computer-usable, for example computer-readable data storage medium can for example be, but is not limited to, an electronic, magnetic, optical, electromagnetic, infrared or semiconductor system, apparatus or device or a medium of propagation such as for example the Internet. The computer-usable or computer-readable data storage medium could even for example be paper or another suitable medium onto which the program is printed, since the program could be electronically captured, for example by optically scanning the paper or other suitable medium, and then compiled, interpreted or otherwise processed in a suitable manner. The data storage medium is preferably a non-volatile data storage medium. The computer program product and any software and/or hardware described here form the various means for performing the functions of the invention in the example embodiments. The computer and/or data processing device can for example include a guidance information device which includes means for outputting guidance information. The guidance information can be outputted, for example to a user, visually by a visual indicating means (for example, a monitor and/or a lamp) and/or acoustically by an acoustic indicating means (for example, a loudspeaker and/or a digital speech output device) and/or tactilely by a tactile indicating means (for example, a vibrating element or a vibration element incorporated into an instrument). For the purpose of this document, a computer is a technical computer which for example comprises technical, for example tangible components, for example mechanical and/or electronic components. Any device mentioned as such in this document is a technical and for example tangible device.

### Acquiring Data

The expression “acquiring data” for example encompasses (within the framework of a computer implemented method) the scenario in which the data are determined by the computer implemented method or program. Determining data for example encompasses measuring physical quantities and transforming the measured values into data, for example digital data, and/or computing (and e.g. outputting) the data by means of a computer and for example within the framework of the method in accordance with the invention. A step of “determining” as described herein for example comprises or consists of issuing a command to perform the determination described herein. For example, the step comprises or consists of issuing a command to cause a computer, for example a remote computer, for example a remote server, for example in the cloud, to perform the determination. Alternatively or additionally, a step of “determination” as described herein for example comprises or consists of receiving the data resulting from the determination described herein, for example receiving the resulting data from the remote computer, for example from that remote computer which has been caused to perform the determination. The meaning of “acquiring data” also for example encompasses the scenario in which the data are received or retrieved by (e.g. input to) the computer implemented method or program, for example from another program, a previous method step or a data storage medium, for example for further processing by the computer implemented method or program. Generation of the data to be acquired may but need not be part of the method in accordance with the invention. The expression “acquiring data” can therefore also for example mean waiting to receive data and/or receiving the data. The received data can for example be inputted via an interface. The expression “acquiring data” can also mean that the computer implemented method or program performs steps in order to (actively) receive or retrieve the data from a data source, for instance a data storage medium (such as for example a ROM, RAM, database, hard drive, etc.), or via the interface (for instance, from another computer or a network). The data acquired by the disclosed method or device, respectively, may be acquired from a database located in a data storage device which is operably to a computer for data transfer between the database and the computer, for example from the database to the computer. The computer acquires the data for use as an input for steps of determining data. The determined data can be output again to the same or another database to be stored for later use. The database or database used for implementing the disclosed method can be located on network data storage device or a network server (for example, a cloud data storage device or a cloud server) or a local data storage device (such as a mass storage device operably connected to at least one computer executing the disclosed method). The data can be made “ready for use” by performing an additional step before the acquiring step. In accordance with this additional step, the data are generated in order to be acquired. The data are for example detected or captured (for example by an analytical device). Alternatively or additionally, the data are inputted in accordance with the additional step, for instance via interfaces. The data generated can for example be inputted (for instance into the computer). In accordance with the additional step (which precedes the acquiring step), the data can also be provided by performing the additional step of storing the data in a data storage medium (such as for example a ROM, RAM, CD and/or hard drive), such that they are ready for use within the framework of the method or program in accordance with the invention. The step of “acquiring data” can therefore also involve commanding a device to obtain and/or provide the data to be acquired. In particular, the acquiring step does not involve an invasive step which would represent a substantial physical interference with the body, requiring professional medical expertise to be carried out and entailing a substantial health risk even when carried out with the required professional care and expertise. In particular, the step of acquiring data, for example determining data, does not involve a surgical step and in particular does not involve a step of treating a human or animal body using surgery or therapy. In order to distinguish the different data used by the present method, the data are denoted (i.e. referred to) as “XY data” and the like and are defined in terms of the information which they describe, which is then preferably referred to as “XY information” and the like.

### Registering

The n-dimensional image of a body is registered when the spatial location of each point of an actual object within a space, for example a body part in an operating theatre, is assigned an image data point of an image (CT, MR, etc.) stored in a navigation system.

### Image Registration

Image registration is the process of transforming different sets of data into one co-ordinate system. The data can be multiple photographs and/or data from different sensors, different times or different viewpoints. It is used in computer vision, medical imaging and in compiling and analysing images and data from satellites. Registration is necessary in order to be able to compare or integrate the data obtained from these different measurements.

### Marker

It is the function of a marker to be detected by a marker detection device (for example, a camera or an ultrasound receiver or analytical devices such as CT or MRI devices) in such a way that its spatial position (i.e. its spatial location and/or alignment) can be ascertained. The detection device is for example part of a navigation system. The markers can be active markers. An active marker can for example emit electromagnetic radiation and/or waves which can be in the infrared, visible and/or ultraviolet spectral range. A marker can also however be passive, i.e. can for example reflect electromagnetic radiation in the infrared, visible and/or ultraviolet spectral range or can block x-ray radiation. To this end, the marker can be provided with a surface which has corresponding reflective properties or can be made of metal in order to block the x-ray radiation. It is also possible fora marker to reflect and/or emit electromagnetic radiation and/or waves in the radio frequency range or at ultrasound wavelengths. A marker preferably has a spherical and/or spheroid shape and can therefore be referred to as a marker sphere; markers can however also exhibit a cornered, for example cubic, shape.

### Marker Device

A marker device can for example be a reference star or a pointer or a single marker or a plurality of (individual) markers which are then preferably in a predetermined spatial relationship. A marker device comprises one, two, three or more markers, wherein two or more such markers are in a predetermined spatial relationship. This predetermined spatial relationship is for example known to a navigation system and is for example stored in a computer of the navigation system.

In another embodiment, a marker device comprises an optical pattern, for example on a two-dimensional surface. The optical pattern might comprise a plurality of geometric shapes like circles, rectangles and/or triangles. The optical pattern can be identified in an image captured by a camera, and the position of the marker device relative to the camera can be determined from the size of the pattern in the image, the orientation of the pattern in the image and the distortion of the pattern in the image. This allows determining the relative position in up to three rotational dimensions and up to three translational dimensions from a single two-dimensional image.

The position of a marker device can be ascertained, for example by a medical navigation system. If the marker device is attached to an object, such as a bone or a medical instrument, the position of the object can be determined from the position of the marker device and the relative position between the marker device and the object. Determining this relative position is also referred to as registering the marker device and the object. The marker device or the object can be tracked, which means that the position of the marker device or the object is ascertained twice or more over time.

### Reference Star

A “reference star” refers to a device with a number of markers, advantageously three markers, attached to it, wherein the markers are (for example detachably) attached to the reference star such that they are stationary, thus providing a known (and advantageously fixed) position of the markers relative to each other. The position of the markers relative to each other can be individually different for each reference star used within the framework of a surgical navigation method, in order to enable a surgical navigation system to identify the corresponding reference star on the basis of the position of its markers relative to each other. It is therefore also then possible for the objects (for example, instruments and/or parts of a body) to which the reference star is attached to be identified and/or differentiated accordingly. In a surgical navigation method, the reference star serves to attach a plurality of markers to an object (for example, a bone or a medical instrument) in order to be able to detect the position of the object (i.e. its spatial location and/or alignment). Such a reference star for example features a way of being attached to the object (for example, a clamp and/or a thread) and/or a holding element which ensures a distance between the markers and the object (for example in order to assist the visibility of the markers to a marker detection device) and/or marker holders which are mechanically connected to the holding element and which the markers can be attached to.

### Navigation System

The present invention is also directed to a navigation system for computer-assisted surgery. This navigation system preferably comprises the aforementioned computer for processing the data provided in accordance with the computer implemented method as described in any one of the embodiments described herein. The navigation system preferably comprises a detection device for detecting the position of detection points which represent the main points and auxiliary points, in order to generate detection signals and to supply the generated detection signals to the computer, such that the computer can determine the absolute main point data and absolute auxiliary point data on the basis of the detection signals received. A detection point is for example a point on the surface of the anatomical structure which is detected, for example by a pointer. In this way, the absolute point data can be provided to the computer. The navigation system also preferably comprises a user interface for receiving the calculation results from the computer (for example, the position of the main plane, the position of the auxiliary plane and/or the position of the standard plane). The user interface provides the received data to the user as information. Examples of a user interface include a display device such as a monitor, or a loudspeaker. The user interface can use any kind of indication signal (for example a visual signal, an audio signal and/or a vibration signal). One example of a display device is an augmented reality device (also referred to as augmented reality glasses) which can be used as so-called “goggles” for navigating. A specific example of such augmented reality glasses is Google Glass (a trademark of Google, Inc.). An augmented reality device can be used both to input information into the computer of the navigation system by user interaction and to display information outputted by the computer.

The invention also relates to a navigation system for computer-assisted surgery, comprising:
a computer for processing the absolute point data and the relative point data;
a detection device for detecting the position of the main and auxiliary points in order to generate the absolute point data and to supply the absolute point data to the computer;
a data interface for receiving the relative point data and for supplying the relative point data to the computer; and
a user interface for receiving data from the computer in order to provide information to the user, wherein the received data are generated by the computer on the basis of the results of the processing performed by the computer.

### Surgical Navigation System

A navigation system, such as a surgical navigation system, is understood to mean a system which can comprise: at least one marker device; a transmitter which emits electromagnetic waves and/or radiation and/or ultrasound waves; a receiver which receives electromagnetic waves and/or radiation and/or ultrasound waves; and an electronic data processing device which is connected to the receiver and/or the transmitter, wherein the data processing device (for example, a computer) for example comprises a processor (CPU) and a working memory and advantageously an indicating device for issuing an indication signal (for example, a visual indicating device such as a monitor and/or an audio indicating device such as a loudspeaker and/or a tactile indicating device such as a vibrator) and a permanent data memory, wherein the data processing device processes navigation data forwarded to it by the receiver and can advantageously output guidance information to a user via the indicating device. The navigation data can be stored in the permanent data memory and for example compared with data stored in said memory beforehand.

### Analytical Devices

The movements of the treatment body parts are for example due to movements which are referred to in the following as “vital movements”. Reference is also made in this respect to EP 2 189 943 A1 and EP 2 189 940 A1, also published as US 2010/0125195 A1 and US 2010/0160836 A1, respectively, which discuss these vital movements in detail. In order to determine the position of the treatment body parts, analytical devices such as x-ray devices, CT devices or MRT devices (also referred to as imaging device(s)) are used to generate analytical images (such as x-ray images or MRT images) of the body. For example, analytical devices are constituted to perform medical imaging methods. Analytical devices for example use medical imaging methods and are for example devices for analysing a patient's body, for instance by using waves and/or radiation and/or energy beams, for example electromagnetic waves and/or radiation, ultrasound waves and/or particles beams. Analytical devices are for example devices which generate images (for example, two-dimensional or three-dimensional images) of the patient's body (and for example of internal structures and/or anatomical parts of the patient's body) by analysing the body. Analytical devices are for example used in medical diagnosis, for example in radiology. However, it can be difficult to identify the treatment body part within the analytical image. It can for example be easier to identify an indicator body part which correlates with changes in the position of the treatment body part and for example the movement of the treatment body part. Tracking an indicator body part thus allows a movement of the treatment body part to be tracked on the basis of a known correlation between the changes in the position (for example the movements) of the indicator body part and the changes in the position (for example the movements) of the treatment body part. As an alternative to or in addition to tracking indicator body parts, marker devices (which can be used as an indicator and thus referred to as “marker indicators”) can be tracked using marker detection devices. The position of the marker indicators has a known (predetermined) correlation with (for example, a fixed relative position relative to) the position of indicator structures (such as the thoracic wall, for example true ribs or false ribs, or the diaphragm or intestinal walls, etc.) which for example change their position due to vital movements.

### Treatment Beam

The present invention relates to the field of controlling a treatment beam. The treatment beam treats body parts which are to be treated and which are referred to in the following as “treatment body parts”. These body parts are for example parts of a patient's body, i.e. anatomical body parts.

The present invention relates to the field of medicine and for example to the use of beams, such as radiation beams, to treat parts of a patient's body, which are therefore also referred to as treatment beams. A treatment beam treats body parts which are to be treated and which are referred to in the following as “treatment body parts”. These body parts are for example parts of a patient's body, i.e. anatomical body parts. Ionising radiation is for example used for the purpose of treatment. For example, the treatment beam comprises or consists of ionising radiation. The ionizing radiation comprises or consists of particles (for example, sub-atomic particles or ions) or electromagnetic waves which are energetic enough to detach electrons from atoms or molecules and so ionize them. Examples of such ionizing radiation include x-rays, high-energy particles (high-energy particle beams) and/or ionizing radiation emitted from a radioactive element. The treatment radiation, for example the treatment beam, is for example used in radiation therapy or radiotherapy, such as in the field of oncology. For treating cancer in particular, parts of the body comprising a pathological structure or tissue such as a tumor are treated using ionizing radiation. The tumor is then an example of a treatment body part.

The treatment beam is preferably controlled such that it passes through the treatment body part. However, the treatment beam can have a negative effect on body parts outside the treatment body part. These body parts are referred to here as “outside body parts”. Generally, a treatment beam has to pass through outside body parts in order to reach and so pass through the treatment body part.

### Arrangement of Treatment Beams

A treatment body part can be treated by one or more treatment beams issued from one or more directions at one or more times. The treatment by means of the at least one treatment beam thus follows a particular spatial and temporal pattern. The term “beam arrangement” is then used to cover the spatial and temporal features of the treatment by means of the at least one treatment beam. The beam arrangement is an arrangement of at least one treatment beam.

The “beam positions” describe the positions of the treatment beams of the beam arrangement. The arrangement of beam positions is referred to as the positional arrangement. A beam position is preferably defined by the beam direction and additional information which allows a specific location, for example in three-dimensional space, to be assigned to the treatment beam, for example information about its co-ordinates in a defined co-ordinate system. The specific location is a point, preferably a point on a straight line. This line is then referred to as a “beam line” and extends in the beam direction, for example along the central axis of the treatment beam. The defined co-ordinate system is preferably defined relative to the treatment device or relative to at least a part of the patient's body. The positional arrangement comprises and for example consists of at least one beam position, for example a discrete set of beam positions (for example, two or more different beam positions), or a continuous multiplicity (manifold) of beam positions.

For example, one or more treatment beams adopt(s) the treatment beam position(s) defined by the positional arrangement simultaneously or sequentially during treatment (for example sequentially if there is only one beam source to emit a treatment beam). If there are several beam sources, it is also possible for at least a subset of the beam positions to be adopted simultaneously by treatment beams during the treatment. For example, one or more subsets of the treatment beams can adopt the beam positions of the positional arrangement in accordance with a predefined sequence. A subset of treatment beams comprises one or more treatment beams. The complete set of treatment beams which comprises one or more treatment beams which adopt(s) all the beam positions defined by the positional arrangement is then the beam arrangement.

### Imaging Methods

In the field of medicine, imaging methods (also called imaging modalities and/or medical imaging modalities) are used to generate image data (for example, two-dimensional or three-dimensional image data) of anatomical structures (such as soft tissues, bones, organs, etc.) of the human body, for example using (an) “imaging device(s)”. The term “medical imaging methods” is understood to mean (advantageously apparatus-based) imaging methods (for example so-called medical imaging modalities and/or radiological imaging methods) such as for instance computed tomography (CT) and cone beam computed tomography (CBCT, such as volumetric CBCT), x-ray tomography, magnetic resonance tomography (MRT or MRI), conventional x-ray, sonography and/or ultrasound examinations, and positron emission tomography. For example, the medical imaging methods are performed by the analytical devices. Examples for medical imaging modalities applied by medical imaging methods are: X-ray radiography, magnetic resonance imaging, medical ultrasonography or ultrasound, endoscopy, elastography, electrography, tactile imaging, thermography, medical photography and nuclear medicine functional imaging techniques such as positron emission tomography (PET) and Single-photon emission computed tomography (SPECT), as mentioned by Wikipedia.

The image data thus generated is also termed “medical imaging data”. Analytical devices for example are used to generate the image data in apparatus-based imaging methods. The imaging methods are for example used for medical diagnostics, to analyze the anatomical body in order to generate images which are described by the image data. The imaging methods are also for example used to detect pathological changes in the human body. However, some of the changes in the anatomical structure, such as the pathological changes in the structures (tissue), may not be detectable and for example may not be visible in the images generated by the imaging methods. A tumor represents an example of a change in an anatomical structure. If the tumor grows, it may then be said to represent an expanded anatomical structure. This expanded anatomical structure may not be detectable; for example, only a part of the expanded anatomical structure may be detectable. Primary/high-grade brain tumors are for example usually visible on MRI scans when contrast agents are used to infiltrate the tumor. MRI scans represent an example of an imaging method. In the case of MRI scans of such brain tumors, the signal enhancement in the MRI images (due to the contrast agents infiltrating the tumor) is considered to represent the solid tumor mass. Thus, the tumor is detectable and for example discernible in the image generated by the imaging method. In addition to these tumors, referred to as “enhancing” tumors, it is thought that approximately 10% of brain tumors are not discernible on a scan and are for example not visible to a user looking at the images generated by the imaging method.

### Mapping

Mapping describes a transformation (for example, linear transformation) of an element (for example, a pixel or voxel), for example the position of an element, of a first data set in a first coordinate system to an element (for example, a pixel or voxel), for example the position of an element, of a second data set in a second coordinate system (which may have a basis which is different from the basis of the first coordinate system). In one embodiment, the mapping is determined by comparing (for example, matching) the color values (for example grey values) of the respective elements by means of an elastic or rigid fusion algorithm. The mapping is embodied for example by a transformation matrix (such as a matrix defining an affine transformation).

### Elastic Fusion, Image Fusion/Morphing, Rigid

Image fusion can be elastic image fusion or rigid image fusion. In the case of rigid image fusion, the relative position between the pixels of a 2D image and/or voxels of a 3D image is fixed, while in the case of elastic image fusion, the relative positions are allowed to change.

In this application, the term “image morphing” is also used as an alternative to the term “elastic image fusion”, but with the same meaning.

Elastic fusion transformations (for example, elastic image fusion transformations) are for example designed to enable a seamless transition from one dataset (for example a first dataset such as for example a first image) to another dataset (for example a second dataset such as for example a second image). The transformation is for example designed such that one of the first and second datasets (images) is deformed, for example in such a way that corresponding structures (for example, corresponding image elements) are arranged at the same position as in the other of the first and second images. The deformed (transformed) image which is transformed from one of the first and second images is for example as similar as possible to the other of the first and second images. Preferably, (numerical) optimization algorithms are applied in order to find the transformation which results in an optimum degree of similarity. The degree of similarity is preferably measured by way of a measure of similarity (also referred to in the following as a “similarity measure”). The parameters of the optimization algorithm are for example vectors of a deformation field. These vectors are determined by the optimization algorithm in such a way as to result in an optimum degree of similarity. Thus, the optimum degree of similarity represents a condition, for example a constraint, for the optimization algorithm. The bases of the vectors lie for example at voxel positions of one of the first and second images which is to be transformed, and the tips of the vectors lie at the corresponding voxel positions in the transformed image. A plurality of these vectors is preferably provided, for instance more than twenty or a hundred or a thousand or ten thousand, etc. Preferably, there are (other) constraints on the transformation (deformation), for example in order to avoid pathological deformations (for instance, all the voxels being shifted to the same position by the transformation). These constraints include for example the constraint that the transformation is regular, which for example means that a Jacobian determinant calculated from a matrix of the deformation field (for example, the vector field) is larger than zero, and also the constraint that the transformed (deformed) image is not self-intersecting and for example that the transformed (deformed) image does not comprise faults and/or ruptures. The constraints include for example the constraint that if a regular grid is transformed simultaneously with the image and in a corresponding manner, the grid is not allowed to interfold at any of its locations. The optimizing problem is for example solved iteratively, for example by means of an optimization algorithm which is for example a first-order optimization algorithm, such as a gradient descent algorithm. Other examples of optimization algorithms include optimization algorithms which do not use derivations, such as the downhill simplex algorithm, or algorithms which use higher-order derivatives such as Newton-like algorithms. The optimization algorithm preferably performs a local optimization. If there is a plurality of local optima, global algorithms such as simulated annealing or generic algorithms can be used. In the case of linear optimization problems, the simplex method can for instance be used.

In the steps of the optimization algorithms, the voxels are for example shifted by a magnitude in a direction such that the degree of similarity is increased. This magnitude is preferably less than a predefined limit, for instance less than one tenth or one hundredth or one thousandth of the diameter of the image, and for example about equal to or less than the distance between neighboring voxels. Large deformations can be implemented, for example due to a high number of (iteration) steps.

The determined elastic fusion transformation can for example be used to determine a degree of similarity (or similarity measure, see above) between the first and second datasets (first and second images). To this end, the deviation between the elastic fusion transformation and an identity transformation is determined. The degree of deviation can for instance be calculated by determining the difference between the determinant of the elastic fusion transformation and the identity transformation. The higher the deviation, the lower the similarity, hence the degree of deviation can be used to determine a measure of similarity.

A measure of similarity can for example be determined on the basis of a determined correlation between the first and second datasets.

### BRIEF DESCRIPTION OF THE DRAWINGS

In the following, the invention is described with reference to the appended figures which give background explanations and represent specific embodiments of the invention. The scope of the invention is however not limited to the specific features disclosed in the context of the figures, wherein

FIG. 1 illustrates the method according to the first aspect of the invention;

FIG. 2 is a first schematic illustration of an example of the system according to the fifth aspect;

FIG. 3 is a second schematic illustration of an example of the system according to the fifth aspect;

FIG. 4 is a third schematic illustration of the system according to the fifth aspect.

### DESCRIPTION OF EMBODIMENTS

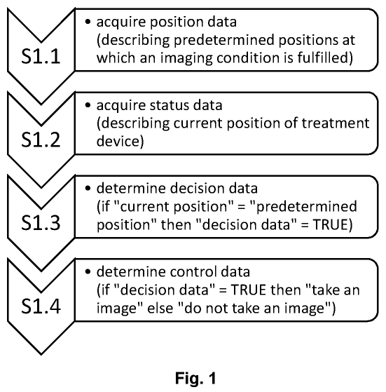

FIG. 1 illustrates the basic steps of the method according to the first aspect, in which step S1.1 encompasses acquiring the position data, step S1.2 encompasses acquiring the status data, step S1.3 encompasses determining the decision data and step S1.4 encompasses determining the control data.

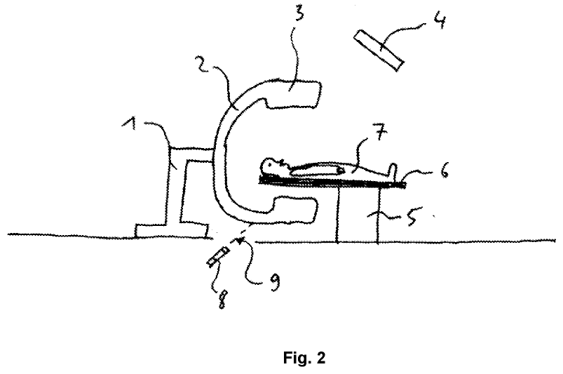

FIG. 2 is a first schematic illustration of an example of the system according to the fifth aspect. In this example, the system comprises a treatment system comprising a stand 1, a gantry 2 and a treatment device 3. A patient 7 is positioned on a patient couch 6 which is placed on a stand 5. An imaging unit comprising an emitter 8 and a receiver 4 has a line of sight indicated by dashed line 9. In the shown configuration, the gantry 2 obstructs the line of sight, i.e. lies between the emitter 8 and the receiver 4 (no free line of sight). In this case, the gantry 2 would be visible in an acquired image and for example cover relevant portions of anatomical body parts of the patient 7 which are to be imaged, for example for surgical navigation.

In the example shown in FIG. 2, the current position of the treatment device 3 described by the status data (which is acquired in step S1.2) does not correspond to a predetermined position described by the position data (which is acquired in step S1.1), in case the imaging condition which needs to be fulfilled at the predetermined position is that the imaging unit has a free line of sight. Thus, the decision data (determined in step S1.3) in this case describes that no image shall be taken by the imaging unit. Therefore, the control data (determined in step S1.4) describes a control signal for controlling the imaging device (and thus also the imaging unit included in the imaging device) not to take an image.

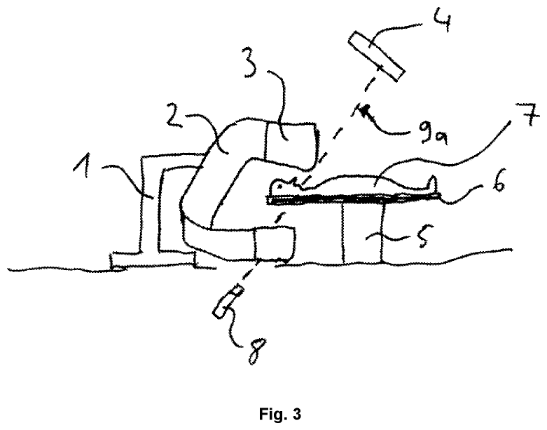

FIG. 3 is a second schematic illustration of an example of the system according to the fifth aspect. This example corresponds to the example described for FIG. 2. However, here the gantry 2 and therefore also the treatment device 3 which is attached to the gantry are positioned differently. As can be seen, the gantry is rotated around an axis which runs in superior—inferior direction of the patient. The gantry may of course also be positioned differently. In the shown example, the imaging unit now has a free line of sight indicated by dashed line 9a. In particular, the gantry 2 does not obstruct the line of sight of the imaging unit. Only the patient 7 and the patient couch 6 are in the line of sight. As described above, this is no reason to prevent an image from being taken, i.e. this configuration is the normal imaging arrangement used to capture an image of relevant anatomical body parts of the patient 7.

In the example shown in FIG. 3, the current position of the treatment device 3 described by the status data (acquired in step S1.2) corresponds to a predetermined position described by the position data (acquired in step S1.1), in case the imaging condition which needs to be fulfilled at the predetermined position is that the imaging unit has a free line of sight. Thus, the decision data (determined in step S1.3) in this case describes that an image shall be taken by the imaging unit. Therefore, the control data (determined in step S1.4) describes a control signal for controlling the imaging device (and thus also the imaging unit included in the imaging device) to take an image.

In the example shown in FIGS. 2 and 3 only one imaging device is depicted. However, there may be two (or more) imaging units included in the imaging device. In one example, the imaging device includes two imaging units. In this example, the imaging condition is met in case each of the two imaging units has a free line of sight, i.e. in case a stereo-image can be acquired without any unwanted devices lying in the line of sight.

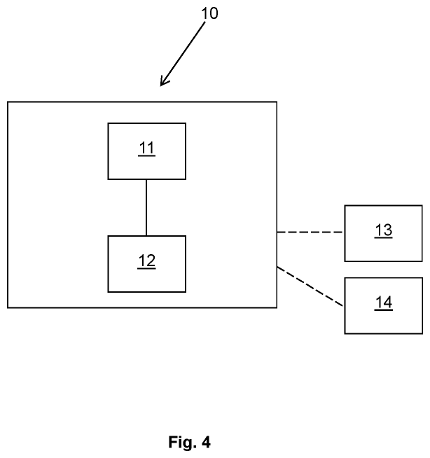

FIG. 4 is a second schematic illustration of the system 10 according to the fifth aspect. The system is in its entirety identified by reference sign 10 and comprises a computer 11, an electronic data storage device (such as a hard disc) 12 for storing at least the patient data. The system may further include the treatment device 13 (such as a radiation treatment apparatus) and the imaging device 14. The components of the medical system 10 have the functionalities and properties explained above with regard to the fifth aspect of this disclosure.

In the following, further advantageous embodiments and advantages of the present invention are described.

For example, an imaging condition can be a certain point in time (e.g. a periodic point in time such as every 10 seconds). The imaging condition can also be that a dose is applied (e.g. obtaining an image of high value. The imaging condition can also be that during a given time period before the imaging condition is met, the patient support device was moved (e.g. after a couch kick (non-coplanar field)).

The imaging condition can also be a clear (free) line of sight (unobstructed line of sight) of one or two imaging units. For example, in case of a stereoscopic imaging system comprising two imaging units, at certain gantry angle segments (at certain positions of the treatment device) a stereo view is possible, i.e. both imaging units have a free line of sight. Also, at certain segments (at certain positions of the treatment device) a first of the two imaging units has a clear (free) line of sight, whilst at (other and/or the same) certain segments (certain positions of the treatment device) the second of the two imaging units has a clear (free) line of sight.

As mentioned for the stereoscopic imaging system, the gantry angles in combination with the positional arrangement of the imaging system and the treatment device define whether there is a free line of sight or not. In other systems which only include a single imaging unit, the imaging unit moves with the gantry (and therefore with the treatment device). Therefore, in this case, images can be acquired from two sufficiently different line of sights (pseudo-3D). The closer two lines of sight are to perpendicularity the higher the value regarding reconstructability.

Other systems such comprise a surface camera system (imaging device) consisting of three cameras (imaging units). The systems may comprise surface cameras and/or thermal cameras. The three cameras are required to ensure a free line of sight at different positions of the gantry (and thus at different positions of the treatment device). Alternatively or additionally, the invention as described herein can be used with such a surface camera system to further enhance the imaging results.

1. A computer implemented method for determining control data describing a control signal for controlling an imaging device to take an image, the method comprising:
acquiring position data that describes at least one predetermined position of a set of predetermined positions of a radiation treatment device, wherein at least one imaging condition is fulfilled when the radiation treatment device is positioned in the at least one predetermined position;
acquiring status data that describes a current position of the radiation treatment device;
determining decision data based on the position data and the status data, the decision data describing that an image shall be taken by the imaging device or that no image shall be taken by the imaging device; and
determining control data based on the decision data, the control data describing a control signal for controlling the imaging device to at least one of take an image or not to take an image.

2. The method according to claim 1, wherein
the imaging device comprises two imaging units,
one of the at least one imaging condition is fulfilled when each of the two imaging units has a free line of sight.

3. The method according to claim 1, wherein
the imaging device comprises an imaging unit,
one of the at least one imaging condition is fulfilled when the imaging unit has a free line of sight.

4. The method according to claim 1,
wherein at least two imaging conditions are fulfilled when the radiation treatment device is positioned in the at least one predetermined position, and
wherein one of the at least two imaging conditions is fulfilled when the at least one predetermined position corresponds to a first position which fulfils a predetermined positional relationship to a second position,
wherein the second position corresponds to another predetermined position of the set of predetermined positions.

5. The method according to claim 1, wherein one of the at least one imaging condition is fulfilled when the predetermined position corresponds to a certain position on a treatment arc defined in a patient treatment plan.

6. The method according to claim 5, wherein the treatment arc is divided into a plurality of arc-segments and the certain position is a position defining a boundary point of an arc-segment.

7. The method according to claim 5, wherein the plurality of arc-segments includes two or more arc-segments of equal size.

8. The method according to claim 1, wherein one of the at least one imaging condition is fulfilled when the predetermined position corresponds to a position at which no treatment beam is to be emitted by the treatment device.

9. The method according to claim 1, wherein one of the at least one imaging condition is fulfilled when the at least one predetermined position corresponds to a position at which the imaging device can take an image.

10. The method according to claim 1, wherein one of the at least one imaging condition is fulfilled when the at least one predetermined position corresponds to a position with a predetermined positional relationship to a collision position, wherein at the collision position the imaging device collides with another device.

11. The method according to claim 1, wherein at least two imaging conditions are fulfilled when the radiation treatment device is positioned in the at least one predetermined position.

12. A non-transitory, computer-readable storage medium having stored thereon computer-executable instructions for a program which, when running on a computer or when loaded onto a computer, causes the computer to:
acquire position data that describes at least one predetermined position of a set of predetermined positions of a radiation treatment device, wherein at least one imaging condition is fulfilled when the radiation treatment device is positioned in the at least one predetermined position;
acquire status data that describes a current position of the radiation treatment device;
determine decision data based on the position data and the status data, the decision data describing that an image shall be taken by the imaging device or that no image shall be taken by the imaging device; and
determine control data based on the decision data, the control data describing a control signal for controlling the imaging device to at least one of take an image or not to take an image.

13. A medical system, comprising:
a treatment device;
an imaging device; and
a computer having computer-executable instructions that, when executed, configure the computer to:
acquire position data that describes at least one predetermined position of a set of predetermined positions of the treatment device, wherein at least one imaging condition is fulfilled when the treatment device is positioned in the at least one predetermined position;
acquire status data that describes a current position of the treatment device:
determine decision data based on the position data and the status data, the decision data describing that an image shall be taken by the imaging device or that no image shall be taken by the imaging device; and
determine control data based on the decision data, the control data describing a control signal for controlling the imaging device to at least one of take an image or not to take an image.

14. The medical system of claim 13, wherein the computer is operably coupled to the treatment device to acquire the status data and to the imaging device to control the imaging device to take the image based on the control signal.

15. (canceled)

16. The medical system of claim 13, wherein the imaging device includes an imaging unit and the at least one imaging condition is fulfilled when the imaging unit has a free line of sight.

17. The medical system of claim 13, wherein at least two imaging conditions are fulfilled when the treatment device is positioned in the at least one predetermined position, one of the at least two imaging conditions is fulfilled when the at least one predetermined position corresponds to a first position having a predetermined positional relationship to a second position, the second position corresponds to another predetermined position of the set of predetermined positions.

18. The medical system of claim 13, wherein the at least one imaging condition is fulfilled when the predetermined position corresponds to a certain position on a treatment arc defined in a patient treatment plan.

19. The medical system of claim 13, wherein the treatment arc is divided into a plurality of arc-segments and the certain position is a position defining a boundary point of an arc-segment.

20. The medical system of claim 13, wherein the at least one imaging condition is fulfilled when the at least one predetermined position corresponds to a position at which the imaging device can take an image.

21. The medical system of claim 20, wherein the position at which the imaging device can take an image includes at least one of a position at which the imaging device does not collide with another device or a position at which components of the imaging device are arranged in an imaging position.
